(19) United States
(12) Patent Application Publication
(10) Pub. No.: US 2004/0199246 A1
(43) Pub. Date: Oct. 7, 2004

(54) Expandable stent

(75) Inventors: Michael S. H. Chu, Brookline, MA (US); Alfred P. Intoccia JR., Amherst, NH (US); Kenneth J. Daignault, Holden, MA (US)

Correspondence Address: TESTA, HURWITZ & THIBEAULT, LLP, HIGH STREET TOWER, 125 HIGH STREET, BOSTON, MA, 02110, US

(73) Assignee: SCIMED Life Systems, Inc., Maple Grove, MN

(21) Appl. No.: 10/405,417

(22) Filed: Apr. 2, 2003

(51) Int. Cl. A61F 2/06

(52) U.S. Cl. 623/1.32; 623/1.11; 623/903; 623/1.44

(57) ABSTRACT

An expandable stent designed to maintain a passageway through a body lumen of a patient is disclosed. The expandable stent is a tubular member that includes a thermoplastic material and woven or wound fibers at least partially in contact with the thermoplastic material, such that the thermoplastic material maintains the tubular member in at least one of an expanded state and a collapsed state.

### TECHNICAL FIELD

This invention generally relates to stents and maintaining a body passageway open.

### BACKGROUND INFORMATION

Body lumens are passageways for the transport of fluid within a human body. Some typical examples of body lumens include veins, arteries, ureters, urethras, esophagi, biliary tracts, and bronchi. Due to a number of different medical conditions, these body lumens may become constricted, thereby limiting or preventing the transport of fluid within the body. To alleviate the constriction and return patency to a patient, a medical professional may insert a stent into the patient's body lumen to reinforce and maintain an open passageway therethrough.

Generally, stents need to be expandable and have a high hoop strength, such that a stent placed within a constricted portion of a body lumen will be able to alleviate the constricted passageway and maintain patency therethrough. Stents also need to be flexible and biocompatible, such that the stent may be easily placed and maintained within the patient's body.

Conventional expandable stents are made from metal in order to achieve proper hoop strength. Plastic stents, while less expensive and generally more biocompatible have yet to achieve proper hoop strength, such that prior art expandable plastic stents are unable to adequately maintain patency through a constricted body lumen.

### SUMMARY OF THE INVENTION

The invention generally relates to maintaining open passageways through body lumens. Devices and methods according to the invention are typically used to treat constrictions and or obstructions within body lumens, such as, for example, a patient's ureter, urethra, esophagus, biliary tract, or vasculature. It is an object of the invention to provide the patient with a stent that maintains an open passageway through a constricted or weakened body lumen. It is another object of the invention to provide the patient with a stent that is resistant to migration once positioned within the patient's body lumen.

It is noted initially that the directional terms proximal and distal require a point of reference. As used herein, the point of reference is from the perspective of a medical professional. Therefore, the term distal refers to a direction that points into the body of the patient and away from the medical professional, whereas the term proximal refers to a direction that points out of the patient's body.

In general, in one aspect, the invention features a stent for use within a body lumen of a patient. The stent includes a tubular member defining a lumen that extends at least partially therethrough and includes a distal end, a proximal end, a thermoplastic material, and woven or wound fibers that are at least partially in contact with the thermoplastic material. The thermoplastic material maintains the tubular member in at least one of an expanded state and a collapsed state.

Embodiments of this aspect of the invention can include the following features. In one embodiment, the woven or wound fibers are at least partially embedded within the thermoplastic material. In another embodiment, the woven or wound fibers are at least partially circumscribed by the thermoplastic material. In other embodiments, the woven or wound fibers at least partially circumscribe the thermoplastic material. The woven or wound fibers can be made of nylon filaments, metal, or thermoplastic materials and can have a cross-sectional shape selected from the group consisting of circular, oval, polygonal, such as, for example, rectangular or triangular, and combinations thereof.

In some embodiments, the tubular member when in the expanded state includes an anchor means on one or both of the distal and proximal ends. In one embodiment, the tubular member further includes a therapeutic agent dispersed at least partially within the thermoplastic material. In other embodiments, the tubular member includes a radiopaque material dispersed at least partially within the thermoplastic material. In some embodiments, the tubular member includes a heat or light sensitive glue disposed on an external surface thereof.

In another aspect, the invention relates to a system for maintaining patency through an anatomical lumen. The system includes a tubular member that defines a lumen extending at least partially therethrough and includes a distal end, a proximal end, a thermoplastic material, woven or wound fibers at least partially in contact with the thermoplastic material, and a transitioning means for transitioning the tubular member between a collapsed state and an expanded state. The thermoplastic material of the tubular member maintains the tubular member in at least one of the expanded state and the collapsed state.

Embodiments of this aspect of the invention can include the following features. In one embodiment, the woven or wound fibers are at least partially embedded within the thermoplastic material. In another embodiment, the woven or wound fibers are at least partially circumscribed by the thermoplastic material. In other embodiments, the woven or wound fibers at least partially circumscribe the thermoplastic material.

In one embodiment of the system, the transitioning means includes a temperature controlled spray of fluid. In an alternative embodiment, the transitioning means includes an expandable member, such as an inflatable balloon attached to and in fluid communication with an insertion rod. The insertion rod defines at least one lumen for providing a fluid to the inflatable balloon and the inflatable balloon is designed to be insertable into the lumen of the tubular member. In some embodiments, the inflatable balloon expands the tubular member from the collapsed state to the expanded state when at least partially filled with a fluid at a temperature greater than a transition temperature of the thermoplastic material and the inflatable balloon is in physical contact with the tubular member. In other embodiments, the tubular member can be maintained in the expanded state after at least partially filling the inflatable balloon with a fluid at a temperature less than about the transition temperature. In one embodiment, the insertion rod includes a lumen for providing a heated fluid to the inflatable balloon and a lumen for providing a cooling fluid to the inflatable balloon.

In another aspect, the invention relates to a method of maintaining patency through an anatomical lumen. The method, according to this aspect of the invention, includes providing the anatomical lumen with a tubular member described above, positioning the tubular member within the anatomical lumen, and transitioning the tubular member from the collapsed state to the expanded state. In some embodiments, the transitioning step includes, positioning an expandable member in contact with the tubular member, heating the expandable member to a temperature greater than about a transition temperature of the thermoplastic material, expanding the expandable member to transition the tubular member to the expanded state, and cooling the expandable member such that the temperature of the expandable member is less than about the transition temperature to maintain the tubular member in the expanded state.

Embodiments of this aspect of the invention can include the following features. In one embodiment, the expandable member is an inflatable balloon attached to and in fluid communication with an insertion rod. The insertion rod defines at least one lumen for providing fluid to the inflatable balloon. In other embodiments, the method according to this aspect of the invention can further include the steps of collapsing the expandable member and removing the expandable member from the anatomical lumen.

The foregoing and other objects, aspects, features, and advantages of the invention will become more apparent from the following description and from the claims.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1A is a plan view of one embodiment of a stent in accordance with the invention;

FIG. 1B is an enlarged view of a portion of the stent of FIG. 1A;

FIGS. 2A and 2B are cross-sectional views of alternative constructions of a stent in accordance with the invention;

FIG. 3A is a plan view of the stent of FIG. 1A in an expanded state;

FIG. 3B is an enlarged view of a portion of the stent of FIG. 3A;

FIG. 4 is a plan view of another embodiment of a stent in accordance with the invention in an expanded state;

FIG. 5 is a plan view of another embodiment of a stent in accordance with the invention in an expanded state;

FIG. 6A is a cross-sectional view of the stent of FIG. 5 taken along line 6A-6A;

FIG. 6B is a cross-sectional view of an alternative embodiment of a stent in accordance with the invention;

FIG. 7 is a plan view of one embodiment of a stent in accordance with the invention in a collapsed state together with one embodiment of a transitioning member;

FIG. 8 is a plan view of the stent and the transitioning member of FIG. 7 after transition into an expanded state;

FIG. 9 is a schematic view of the stent and the transitioning member of FIG. 7 being inserted into a patient's urinary system;

FIG. 10 is an enlarged schematic view of a portion of the urinary system labeled B in FIG. 9 with the stent of FIG. 7 properly positioned within the patient's urinary system;

FIG. 11 is an enlarged schematic view of the stent of FIG. 7 being expanded by the transitioning member; and

FIG. 12 is an enlarged schematic view of the stent of FIG. 7 after expansion within the patient's urinary system.

### DESCRIPTION

A medical professional may insert a stent into a patient's body to maintain a passageway through a constricted body lumen, thereby allowing fluids to pass freely therethrough. For example, a male patient afflicted with benign prostatic hyperplasia (BPH) experiences urine retention due to enlargement of the patient's prostate and consequential constriction of his prostatic urethra. A physician will typically insert a stent into the patient's urethra and position the stent such that it reinforces the prostatic urethra to alleviate constriction of this body lumen and to maintain an open passageway for the transport of urine therethrough. Similarly, as further non-restricting examples, the physician can place a stent within a passageway into, out of, or within the patient's heart or in a patient's ureter to alleviate constriction and obstruction of fluid flow.

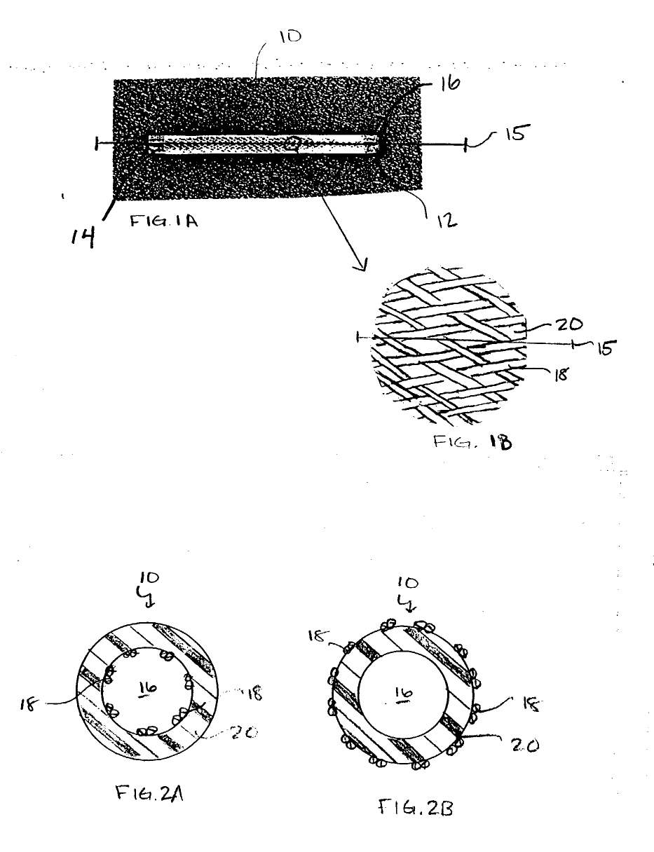

Referring to FIGS. 1A and 1B, a stent 10 is a tubular member, for example a passageway for fluids including a circular, oval, elliptical, or polygonal cross-sectional shape. The stent 10 includes a distal end 12 and a proximal end 14 and defines a lumen 16 that extends within the stent 10 along a longitudinal axis 15 to allow fluids, such as urine or blood to pass therethrough. The stent 10 also includes woven or wound fibers 18 that are at least partially in contact with a thermoplastic material 20, such as, for example, ethylene vinyl acetate, polymethylmethacrylate, polystyrene, and polyethylene terephthalate, or any other suitable biocompatible thermoplastic material.

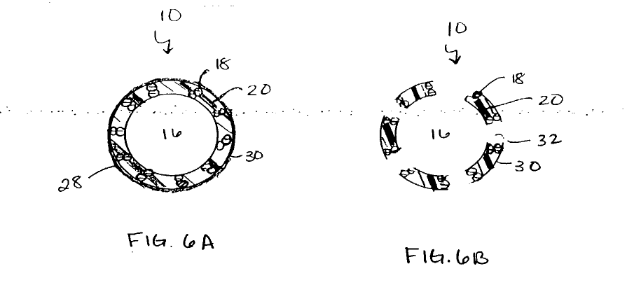

The stent 10 has at least two states, a collapsed state, as shown in FIG. 1A, and an expanded state, as shown in FIG. 3A. The thermoplastic material 20 when heated becomes soft and malleable, such that a diameter of the lumen 16 can be expanded or collapsed. Upon cooling, the thermoplastic material 20 hardens and maintains a current state of the stent 10, i.e., either the collapsed state or the expanded state. The woven or wound fibers 18 are at least partially in contact with the thermoplastic material 20 and thus are maintained in a particular position to achieve the collapsed state or the expanded state when the thermoplastic material 20 is hardened.

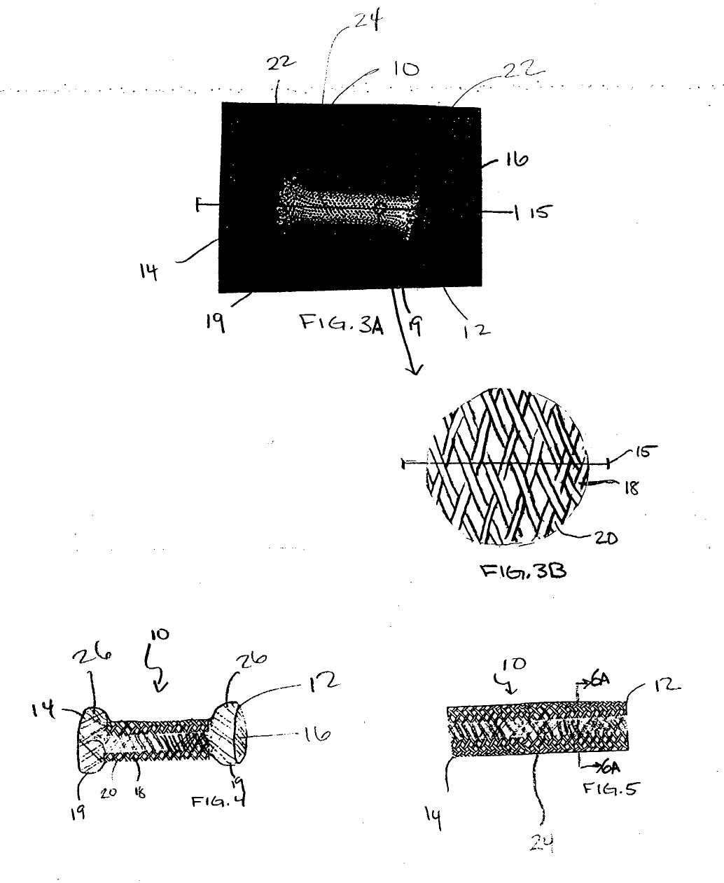

In FIGS. 1A and 1B, the woven or wound fibers 18 are at least partially embedded within the thermoplastic material 20. In other embodiments, for example the embodiments shown in FIGS. 2A and 2B, the woven or wound fibers 18 are at least partially circumscribed by the thermoplastic material 20 or circumscribe the thermoplastic material 20, respectively. Other embodiments are also possible, as long as the woven or wound fibers 18 are at least partially in contact with the thermoplastic material 20.

The woven or wound fibers 18 used to form the stent 10 may have one of a variety of cross-sectional shapes, such as, for example, circular, oval, square, rectangular, triangular, or combinations thereof. In the embodiment shown, the fibers 18 have a circular cross-sectional shape having a cross-sectional area defined by each fiber's diameter. The diameter or thickness, or more generally the cross-sectional area or shape, of the fibers 18 selected for the stent 10 influences the radial strength as well as the flexibility of the stent 10. Thus, for the disclosed embodiment, the diameter of the fibers selected needs to be sufficiently large to assure that proper radial or hoop strength of the stent 10 is achieved to alleviate constriction within a body lumen. At the same time, however, the diameter of the fibers 18 also needs to be sufficiently thin to promote flexibility of the stent 10 to accommodate the patient's anatomy. In one embodiment, the diameter of the round fibers used to form the stent 10 is generally in the range of from about 0.1 millimeters to about 3 millimeters, which corresponds to a cross-sectional area in the range of about 7.9×10⁻³ millimeters² to about 7.1 millimeters². In the disclosed embodiment, the fiber's diameter is 1 millimeter, which corresponds to a cross-sectional area of about 0.79 millimeters².

The woven or wound fibers 18 must be able to move relative to each other when the thermoplastic material 20 is malleable such that the diameter of the stent 10 can expand and contract to reconfigure the stent 10 between the collapsed state shown in FIG. 1A and the expanded state shown in FIG. 3A. In one embodiment, the woven or wound fibers 18 are made of a biocompatible plastic, such as, for example, nylon. In other embodiments, the woven or wound fibers 18 may be made of a metal, such as titanium, a metal alloy, or even a shape memory alloy, such as, for example, a nickel-titanium alloy. In another embodiment, the woven or wound fibers 18 may be made out of a thermoplastic material that is rigid when cool, but softens and becomes malleable with heat. The weave and pitch of the woven or wound fibers 18 can further influence the hoop strength of the stent 10. For example, if the braid of the woven or wound fibers 18 is loose, such that large gaps are created between individual woven or wound fibers 18, the stent 10 will have increased flexibility and decreased hoop strength. Alternatively, if the braid of the woven or wound fibers 18 is tight such that there are substantially no gaps between individual woven or wound fibers 18, the stent 10 will possess increased hoop strength at the expense of some flexibility.

In the collapsed state, as shown in FIGS. 1A and 1B, the fibers 18 are oriented such that the fibers 18 are substantially parallel to the longitudinal axis 15 of the stent 10 i.e., the fibers 18 and the longitudinal axis 15 are offset by an angle of 0 degrees to 20 degrees. Once the thermoplastic material 20 has become malleable, radial expansive forces acting on the stent 10 force the fibers to move relative to each other to a new orientation in which the fibers are approaching 45 degrees to 90 degrees relative to the longitudinal axis 15, as shown in FIGS. 3A and 3B, to form the expanded state. The transition in orientation of the fibers 18 from about 0°-20° to about 45°-90° from the longitudinal axis 15 results in an increase in width or diameter of the stent 10 and a decrease in length of the stent 10.

One advantage of the stent 10 over conventional prior art metal stents is ease of manufacture. The stent 10 can be machine braided and then extruded at least to partially embed the stent 10 with the thermoplastic material 20. The stent 10 can be cut to a desired length using a hot knife to form blunt edges at the distal and proximal ends 12, 14 of the stent. Also, a manufacturer can easily produce the stent 10 in a variety of different diameters, thereby allowing a physician to be able to select an appropriately sized stent without regard to increased manufacturing costs. For example, when treating a constricted prostatic urethra, a stent with a diameter in the expanded state of about 8 mm may be the most appropriate for a particular patient, whereas a stent with a diameter of about 6 mm may be more/appropriate for a different patient.

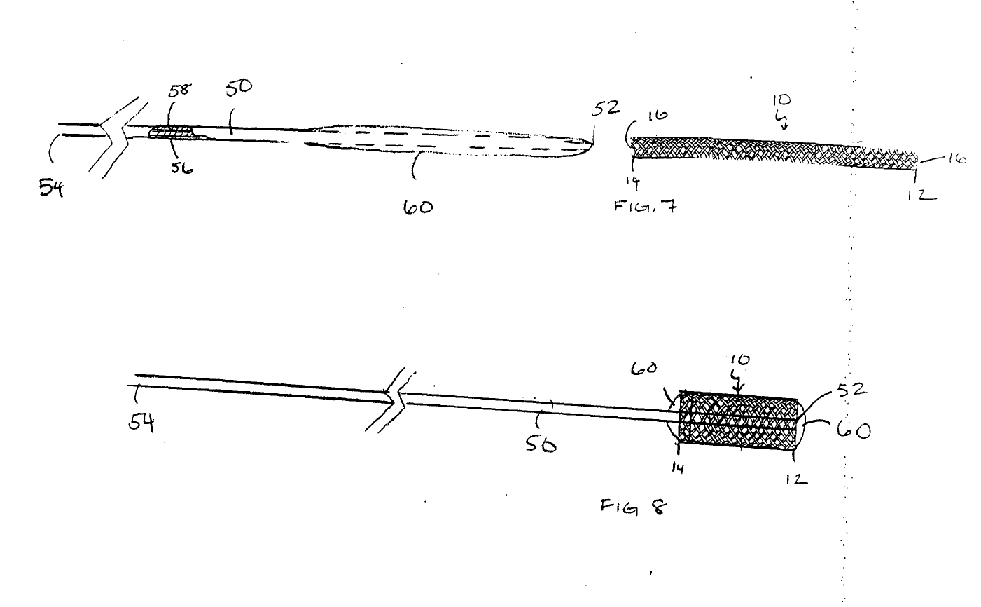

To retain proper positioning of the stent 10 when the stent is left within the patient's body and to inhibit movement of the positioned stent 10, each of the distal end 12 and proximal end 14 may include an anchor means 19 (FIGS. 3 and 4). As depicted in FIG. 3, the distal and proximal ends 12, 14 of the stent 10 in the expanded state are flared, such that the distal and proximal ends 12, 14 have a larger diameter and cross-sectional area 22 then a portion 24 of the stent extending between the distal and proximal ends 12, 14. The flared distal and proximal ends 12, 14 act as anchors to hold the stent 10 in the desired position.

In another embodiment, depicted in FIG. 4, the distal and proximal ends 12, 14 each form a funnel shape 26 when expanded. The funnel shape 26 secures and prevents the stent 10 from migrating from a portion of the body lumen that is to be treated to alleviate constriction. Both the flared distal and proximal ends with larger cross-sectional area 22 and the funnel shape 26 may be formed by varying the braid of the fibers 18 at the distal and proximal ends 12, 14 and/or by using a specially designed inflatable balloon (i.e., a balloon that has a contoured shape or a pre-selected diameter when fully inflated) to expand the stent 10 to a larger extent at the distal and proximal ends 12, 14. In some embodiments, the distal and proximal ends 12, 14 include substantially no fibers 18, thereby allowing the distal and proximal ends 12, 14 to have increased malleability to form the anchor means 19. Also, it should be noted that the length and diameter of the stent 10 and the length and diameter of the anchors at the distal and proximal ends 12,14 may vary in size to suit a particular application and anatomy within the patient's body.

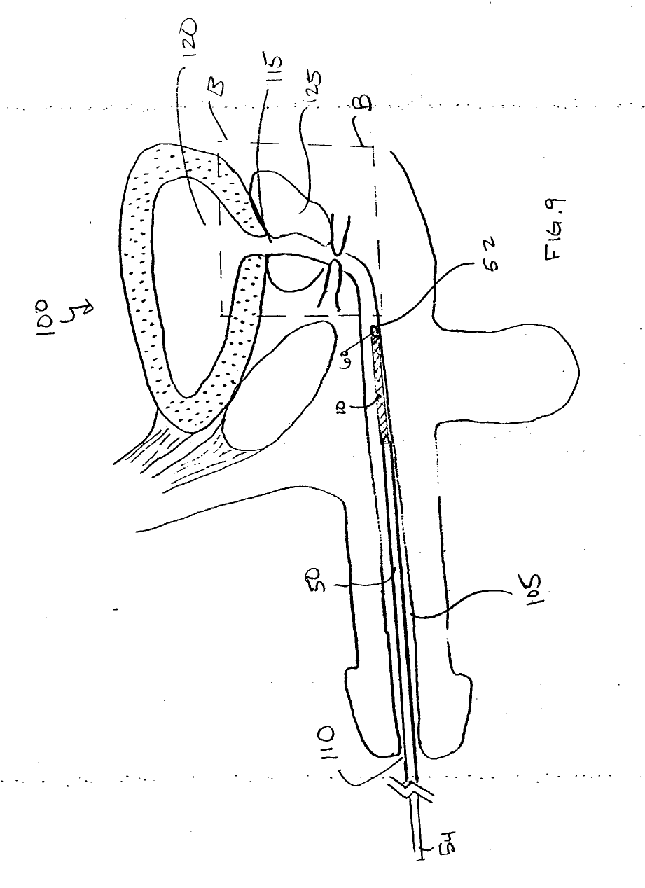

Referring to FIGS. 5, 6A, and 6B, the stent 10 need not have anchor means to prevent migration. As shown in FIG. 6A, a heat or light sensitive glue 28 applied to an external surface 30 of the stent 10 retains the stent 10 in position after placement by the physician. Alternatively, in other embodiments, such as the embodiment depicted in FIG. 6B, tissue ingrowth into the stent 10 can prevent stent migration. In the embodiment shown in FIG. 6B, the woven or wound fibers 18 are partially embedded within the thermoplastic material 20, such that openings 32 are created. After placement of the stent 10 within the patient's body, tissue may grow into these openings 32 and prevent movement of the stent 10 from its intended position. In other embodiments, only the distal and proximal ends 12, 14 include the thermoplastic material 20 and the openings 32 between fibers 18 within the portion 24 between the distal and proximal ends 12, 14 of the stent 10 are available for tissue ingrowth.

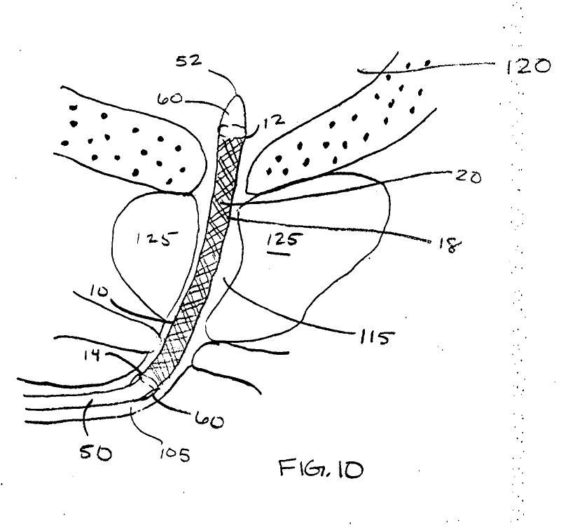

In operation, the physician uses a transitioning means for transitioning the tubular member between the collapsed state and the expanded state. One example of the transitioning means, shown in FIG. 7, is an expandable member, such as, for example, an inflatable balloon 60. An insertion rod 50 attached to the inflatable balloon 60 delivers, positions, and expands the stent 10 from the collapsed state to the expanded state within the patient's body. The insertion rod 50 has a distal end 52 and a proximal end 54, and is sized to fit within the lumen 16 of the stent 10. The insertion rod 50 is made from any biocompatible material that is sufficiently flexible to navigate around natural bends in the patient's anatomy, while simultaneously sufficiently rigid to push the stent 10 through a constricted body lumen. The insertion rod 50 also includes at least one lumen 56 for providing a fluid to the inflatable balloon 60 through one or more balloon inlet and outlet ports.

Prior to insertion into the patient's body, the physician connects the stent 10 and the insertion rod 50 by inserting the distal end 52 of the insertion rod 50 into the lumen 16 of the stent 10. Next, the physician inflates the inflatable balloon 60 just enough to contact and secure the stent 10, in the collapsed state, to the insertion rod 50. With the stent 10 in the collapsed state, the physician inserts and positions the stent 10 within the patient's constricted body lumen. Once the stent is properly positioned, the physician will fill the inflatable balloon 60 with a fluid to transition the stent 10 to the expanded state, as depicted in FIG. 8.

To expand the stent 10, the physician introduces a heated fluid into the inflatable balloon 60 via the at least one lumen 56 within the insertion rod 50. In some cases the physician can use a high-pressure syringe to introduce the heated fluid into the inflatable balloon 60. The heated fluid heats and fills the inflatable balloon 60 and through conduction heats the thermoplastic material 20 of the stent 10. The thermoplastic material 20 becomes malleable and expands with the expanding inflatable balloon 60 once the thermoplastic material has achieved a temperature of or greater than the thermoplastic material's glass transition temperature. Generally, it is advantageous to select a thermoplastic material that has a glass transition temperature above about human body temperature, 98.6° F., so that expansion of the stent 10 can be controlled by the physician. The woven or wound fibers 18 are pushed radially outward by the expanding inflatable balloon 60 and transition from an orientation of about 0°-20° from the longitudinal axis 15 to an orientation approaching 45°-90° from the longitudinal axis 15 to achieve the expanded state.

After expanding the stent 10 to its expanded state, the physician introduces a cooling fluid, i.e., a fluid with a temperature below the glass transition temperature of the thermoplastic material and preferably below human body temperature, into the inflatable balloon 60. The cooling fluid can be introduced into the inflatable balloon 60 via the at least one lumen 56 within the insertion rod 50. Alternatively, the insertion rod 50 can include a second lumen 58 dedicated to providing the cooling fluid to the inflatable balloon 60. In response, the thermoplastic material 20 of the stent 10 hardens, thereby maintaining and holding the fibers 18, and hence the stent 10, in the expanded state (i.e., the fibers are oriented 45°-90° from the longitudinal axis 15). Subsequently, the physician drains the fluid from the inflatable balloon 60, thereby collapsing the inflatable balloon 60, and then removes the insertion rod 50 and the inflatable balloon 60 without disturbing the position of the stent 10. As illustrated in FIG. 8, the stent 10, when in the expanded state, has an increased diameter as compared to the stent 10 in the collapsed state (FIG. 7). It should be noted, however, that due to the expansion of the diameter of the stent 10, the length of the stent 10 in the expanded state is less than the length of the stent 10 in the collapsed state.

Another example of the transitioning means is a temperature controlled spray of fluid, such as heated or cooled water. In this embodiment, the physician expands the stent 10, which is already positioned within the patient's body lumen, by partially filling the body lumen with the heated fluid via the temperature controlled spray of fluid. Hence, the stent 10 is surrounded by a bath of the heated fluid, thereby causing the thermoplastic material 20 to become malleable and expandable. Increased pressure caused by the bath in the patient's body lumen surrounding the stent 10 forces the fibers 18 to transition from the collapsed state to the expanded state. Once the stent 10 has achieved the expanded state (i.e., the fibers 18 are transitioned to an orientation of about 45°-90° from the longitudinal axis 15), the physician can maintain the stent 10 in its current state by introducing the cooling fluid via the temperature controlled spray of fluid.

While the following example generally describes a procedure for positioning the stent 10 within the patient's prostatic urethra, it should be noted that similar processes can be used to place the stent 10 within other constricted body lumens, such as, for example, the ureter or within a passageway through or into or out of the heart.

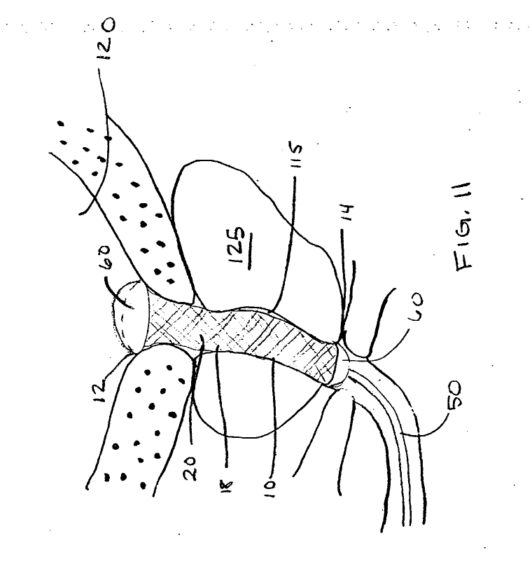

Referring to FIGS. 9 and 10, in one embodiment of an application of placing the stent 10 in a patient's urethra, the physician inserts the stent 10, in the collapsed state and attached to the insertion rod 50, into the patient's urinary system 100 via an external opening 110 to the patient's urethra 105. The physician advances the stent 10 and the insertion rod 50 through the patient's urethra 105 until the stent 10 is located substantially within the prostatic urethra 115 and the distal end 12 of the stent 10 is located near an opening to the patient's bladder 120. So that the physician can confirm proper placement of the stent by radiographic techniques, a small amount of metal or other radiopaque material, such as, for example, bismuth, may be embedded within the thermoplastic material 20 of the stent 10 or alternatively at the distal end 52 of the insertion rod. Other means for ensuring proper placement may be used, such as, for example, ultrasonic guidance or blind placement using a placement balloon at the distal end of the insertion road, which may be inflated independently of the expansion balloon and seated in the bladder neck to position the stent.

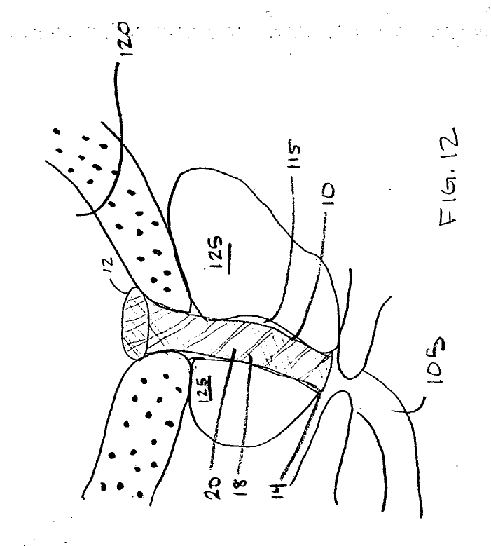

After confirmation of proper placement, the physician introduces the heated fluid to expand both the inflatable balloon 60 and the stent 10, as depicted in FIG. 11. The stent 10 in its expanded state opens up the prostatic urethra 115 and prevents an enlarged prostate 125 from constricting the prostatic urethra 115 and restricting urine flow.

Prior to removing the insertion rod 50, the physician introduces the cooling fluid into the inflatable balloon 60 to harden the thermoplastic material 20, thereby maintaining the stent 10 in the expanded state. Next, the physician drains the fluid from the inflatable balloon 60 to collapse the inflatable balloon prior to removing the insertion rod 50. As depicted in FIG. 12, the stent 10 remains in the expanded state reinforcing the prostatic urethra 115 against collapse from the enlarged prostate 125. The distal end 12 is enlarged to form an anchor to secure the stent 10 in its intended position within the prostatic urethra 115. In some embodiments, medicine and other drugs, e.g., therapeutic agents, may be implanted or dispersed within the thermoplastic material 20 of the stent 10 and delivered through diffusion to the prostatic urethra 115. In other embodiments, therapeutic agents applied to or dispersed on the external surface 30 of the stent 10 are absorbed by the prostatic urethra 115 directly.

At some later time, the physician can remove the stent 10 from the prostatic urethra 115 by inserting the insertion rod 50 and the inflatable balloon 60 into the lumen 16 of the stent 10, filling the inflatable balloon 60 with a heated fluid to soften the thermoplastic material 20, and then draining the heated fluid from the inflatable balloon 60, thereby causing the stent 10 to collapse in response. The heated stent 10 could be collapsed as a function of, for example, the weight of the stent, residual compression of the body lumen, or by some external mechanism. With the stent 10 in the collapsed state, the physician can easily remove the stent 10 from the patient's body with minimal injury.

Variations, modifications, and other implementations of what is described herein will occur to those of ordinary skill in the art without departing from the spirit and the scope of the invention. Accordingly, the invention is not to be defined only by the preceding illustrative description.

What is claimed is:

1. A tubular member including a distal end and a proximal end and defining a lumen extending at least partially therethrough, the tubular member comprising:
a thermoplastic material; and
fibers selected from the group consisting of woven and wound fibers at least partially in contact with the thermoplastic material, the thermoplastic material maintaining the tubular member in at least one of an expanded state and a collapsed state.

2. The tubular member of claim 1 wherein the fibers are at least partially embedded within the thermoplastic material.

3. The tubular member of claim 1 wherein the fibers are at least partially circumscribed by the thermoplastic material.

4. The tubular member of claim 1 wherein the fibers at least partially circumscribe the thermoplastic material.

5. The tubular member of claim 1 wherein the fibers are woven and comprise nylon filaments.

6. The tubular member of claim 1 wherein the fibers are woven comprise a metal.

7. The tubular member of claim 1 wherein the distal end includes an anchor means when in the expanded state.

8. The tubular member of claim 1 wherein the proximal end includes an anchor means when in the expanded state.

9. The tubular member of claim 1 wherein the fibers comprise a cross-sectional shape selected from the group consisting of circular, oval, polygonal, and combinations thereof.

10. The tubular member of claim 1 further comprising a therapeutic agent dispersed at least partially within the thermoplastic material.

11. The tubular member of claim 1 further comprising a radiopaque material dispersed at least partially within the thermoplastic material.

12. The tubular member of claim 1 further comprising a heat sensitive glue disposed on an external surface of the tubular member.

13. The tubular member of claim 1 further comprising a light sensitive glue disposed on an external surface of the tubular member.

14. A system for maintaining patency through an anatomical lumen, the system comprising:
a tubular member including a distal end and a proximal end and defining a lumen extending at least partially therethrough, the tubular member comprising:
a thermoplastic material; and
fibers selected from the group consisting of woven and wound fibers at least partially in contact with the thermoplastic material, the thermoplastic material maintaining the tubular member in at least one of an expanded state and a collapsed state; and
a transitioning means for transitioning the tubular member between the collapsed state and the expanded state.

15. The system of claim 14 wherein the fibers are at least partially embedded within the thermoplastic material.

16. The system of claim 14 wherein the fibers are at least partially circumscribed by the thermoplastic material.

17. The system of claim 14 wherein the fibers at least partially circumscribe the thermoplastic material.

18. The system of claim 14 wherein the transitioning means comprises a temperature controlled spray of fluid.

19. The system of claim 14 wherein the transitioning means comprises an expandable member.

20. The system of claim 19 wherein the expandable member comprises an inflatable balloon attached to and in fluid communication with an insertion rod, the insertion rod defining at least one lumen for providing a fluid to the inflatable balloon, the inflatable balloon being insertable into the lumen of the tubular member.

21. The system of claim 20 wherein the inflatable balloon expands the tubular member from the collapsed state to the expanded state when the inflatable balloon is at least partially filled with a fluid at a temperature greater than about a transition temperature of the thermoplastic material and is in physical contact with the tubular member.

22. The system of claim 21 wherein the tubular member is maintained in the expanded state after at least partially filling the inflatable balloon with a fluid at a temperature less than about the transition temperature.

23. The system of claim 20 wherein the insertion rod includes a lumen for providing a heated fluid to the inflatable balloon and a lumen for providing a cooling fluid to the inflatable balloon.

24. A method of maintaining patency through an anatomical lumen, the method comprising the steps of:
providing the anatomical lumen with a tubular member in a collapsed state, the tubular member including a distal end and a proximal end and defining a lumen extending at least partially therethrough, the tubular member comprising:
a thermoplastic material, and
fibers selected from the group consisting of woven and wound fibers at least partially in contact with the thermoplastic material, the thermoplastic material maintaining the tubular member in at least one of an expanded state and the collapsed state;
positioning the tubular member within the anatomical lumen; and
transitioning the tubular member from the collapsed state to the expanded state.

25. The method of claim 24 wherein transitioning the tubular member comprises:
positioning an expandable member in contact with the tubular member;
heating the expandable member to a temperature greater than about a transition temperature of the thermoplastic material;
expanding the expandable member to transition the tubular member to the expanded state; and
cooling the expandable member such that the temperature of the expandable member is less than about the transition temperature to maintain the tubular member in the expanded position.

26. The method of claim 25 wherein the expandable member is an inflatable balloon attached to and in fluid communication with an insertion rod; the insertion rod defining at least one lumen for providing a fluid to the inflatable balloon.

27. The method of claim 25 further comprising the steps of:
collapsing the expandable member; and
removing the expandable member from the anatomical lumen.
